(19) United States
(12) Patent Application Publication
(10) Pub. No.: US 2026/0215945 A1
(43) Pub. Date: Jul. 30, 2026

(54) Ureteral Guidewire And Stent

(71) Applicant: L and L Medical, LLC, Gainesville, GA (US)

(72) Inventor: Lawrence E. Lykins, Gainesville, GA (US)

(21) Appl. No.: 19/439,348

(22) Filed: Jan. 3, 2026

Related U.S. Application Data
(63) Continuation-in-part of application No. 19/036,849, filed on Jan. 24, 2025, now abandoned.
Application No. 19/036,849 is a continuation of application No. 16/269,455, filed on Feb. 6, 2019, now abandoned.
(60) Provisional application No. 62/709,921, filed on Feb. 6, 2018.

(51) Int. Cl. A61F 2/95; A61B 1/307; A61F 2/86

(52) U.S. Cl. CPC A61F 2/95; A61B 1/307; A61F 2/86

(57) ABSTRACT

A guidewire for use with ureteral stents has an elongated shape with a proximal end and a distal end. The guidewire has indicia along the proximal end to indicate the length of the guidewire that was introduced into the patient. Based on the indicia or the visual inspection, a physician can select the appropriately sized stent to place in the patient.

### CROSS-REFERENCE TO RELATED APPLICATIONS

This application is a continuation-in-part of, and claims priority under 35 USC §120 to, US Patent Application No. 19/036,849 filed 24 January 2025, which US Patent Application No. 17/923,761 is a continuation of, and claims benefit under 35 USC §120 to, US Patent Application No. 16/269,455 filed 6 February 2019, which US Patent Application No. 16/269,455 claims the benefit under 35 USC §119(e) to US Provisional Patent Application No. 62/709,921 filed 6 February 2018, the entirety of each of which is incorporated herein by reference as if fully set forth below.

### STATEMENT REGARDING FEDERALLY SPONSORED RESEARCH OR DEVELOPMENT

Not Applicable

### SEQUENCE LISTING

Not Applicable

### STATEMENT REGARDING PRIOR DISCLOSURES BY THE INVENTOR OR A JOINT INVENTOR

Not Applicable

### BACKGROUND OF THE DISCLOSURE

### 1. Field of the Invention

This disclosure generally relates to urinary catheters and ureteral stents. More particularly, this disclosure relates to guidewires having varying indicium (e.g., mark size markings) along their length and a platinum radiopaque marker 5 centimeters ( cm) from the distal end.

### 2. Description of Related Art

Insertion of double J stent is a very common procedure performed by urologists and physicians. This procedure is often performed to relieve ureteral obstruction due to stones, tumors, ureteral kinks, or stricture on an urgent basis. Double J ureteral stents are small tubes with a 5 cm curl on each end inserted into the ureter to treat or prevent a blockage that prevents the flow or urine from the kidney to the bladder. An increasing number of urologists or physicians are using a flexible cystoscope and local anesthetic for J-stent insertion. In most cases, the procedure is straightforward. After placement of guidewire, the physician pushes the ureteral stent upwardly on the wire until the distal end is in the renal pelvis visualized by fluro x-ray.

Urinary stents typically have a 5 cm bladder curl and a 5 cm distal curl made from a soft material to prevent migration and bladder irritation. If the stent is placed up too far or not far enough, the patient experiences discomfort, which may result in physician return visits or/and replacement of the stent. Patient discomfort from these stents comes from a number of areas. One such area is that the bladder curl can rub and irritate the inside of the bladder, especially the sensitive trigone area. Another such area is that flank pain is perceived by the patient due to the reflux occurring as the bladder voids and urine is pushed up through the stent lumen back into the kidney. Because the curl on the stent is 5 cm the platinum marker on this guidewire is at 5 cm from the distal end and thereby making the correct length stent to be placed accurately.

Presently, when a physician places a stent in a ureter, he/she first passes a radiopaque guidewire up the ureter. When the floppy end of the guidewire reaches the kidney pelvis, it coils in the renal pelvis. This allows the physician to see this on fluoroscopy and know the guidewire is in the kidney pelvis. Presently, when a physician introduces a stent in a ureter over the guidewire, the urologist estimates the length of the stent that is suitable for the patient. This estimation is usually done by assessing the patient’s height, which is only a partial predictor of the appropriately sized stent. For illustration, stents typically come in even numbered lengths, i.e., 20 cm, 22 cm, 24 cm, 26 cm, 28 cm, 30 cm - the physician selects the stent size based on the height of patient, which is probably a good predictor over half of the time. If the urologist places a stent that is too short, the stent retracts up into the ureter, and a separate procedure may be required to get the stent out of the ureter. If the stent is too long, too much of the stent ends up in the bladder, add it irritates the bladder especially the sensitive trigone area causing the patient to have to urinate painfully and frequently, which causes a lot of discomfort to the patient.

Accordingly, there is a need for a technique and device that can improve the selection of a stent, the correct length of stent to place, and the size thereof suitable for a patient. It is to this need, among others, that this invention is directed.

### BRIEF SUMMARY OF THE INVENTION

In an exemplary embodiment, the present invention is a guidewire that has a 5 cm floppy distal end with a radiopaque platinum marker 5 cm from the distal end. The guidewire has indicia along the proximal end to visually indicate the length of guidewire that runs from the UPJ (ureteral pelvic junction) to the ureteral orifice. Based on the indicia visualized on the guidewire the physician can select to within 1 cm the length of stent to place in the ureter.

In another exemplary embodiment, the present invention is a method for inserting a ureteral stent into the ureter in a patient comprising injecting radiopaque contrast into the ureter that shows the renal pelvis by fluro x-ray and the UPJ, introducing into the ureter a guidewire having a platinum marker 5 cm from the distal end and having indicia at the proximal end of the guidewire into the patient, examining the indicia at the ureteral orifice where the ureter enters the bladder, selecting a stent length based on the color of the indicia at the UVO (ureteral orifice) white-22 cm stent, black 24 cm stent, or green 26 cm stent, selecting the stent over the guidewire into the ureter, and removing the guidewire from the stent, leaving the stent in place. Preferably this leaves the stent of correct length to within 1 cm for the patient.

In another exemplary embodiment, the present invention is a guidewire for placement of a ureteral stent comprising a length defined between a proximal end and a distal end, fluoroscopic indicia located between the ends in proximity to the distal end, and spaced a placement distance from the distal end, and visual indica located between the ends in proximity to the proximal end, wherein: the fluoroscopic indicia aids in a proper placement of the ureteral stent by providing a medical professional a fluoroscopic queue that the distal end of the guidewire is sufficiently located within the kidney, that upon delivery of the ureteral stent and then subsequent guidewire withdrawal, a distal curled end of the ureteral stent will remain in the kidney, and wherein the visual indica aiding in the proper placement by providing the medical professional a visual queue indicative of a properly selected length for the ureteral stent, that upon delivery of the ureteral stent of the properly selected length and then subsequent guidewire withdrawal, a proximal curled end of the ureteral stent will remain in the bladder.

In any embodiment, the fluoroscopic indicia can comprise a platinum marker with a placement distance of 5 cm.

In any embodiment, the visual indicia can comprise multiple bands of greater than 1 cm along a length of the guidewire in proximity to the proximal end. In any embodiment, the visual indicia can comprise multiple bands of greater than 2 cm.

In any embodiment, the guidewire can be a metallic wire.

In any embodiment, the visual indicia can comprise one or more colors.

In any embodiment, the distal end of the guidewire can comprise a 5 cm curve.

In any embodiment, the fluoroscopic indicia can comprise a platinum marker with a placement distance of 5 cm, the visual indicia can comprise multiple bands of greater than 1 cm along a length of the guidewire in proximity to the proximal end, the guidewire can comprise a metallic wire, and the distal end of the guidewire can comprise a 5 cm curve.

In another exemplary embodiment, the present invention is a guidewire for placement of a ureteral stent comprising a length defined between a proximal end and a distal end, fluoroscopic indicia located between the ends in proximity to the distal end, and spaced a placement distance from the distal end, and visual indica located between the ends in proximity to the proximal end, and wherein the fluoroscopic indicia aids in a proper placement of the ureteral stent by providing a medical professional a fluoroscopic queue that the distal end of the guidewire is sufficiently located within the kidney, that upon delivery of the ureteral stent and then subsequent guidewire withdrawal, a distal curled end of the ureteral stent will remain in the kidney, and the visual indica aiding in the proper placement by providing the medical professional a visual queue indicative of a properly selected length for the ureteral stent, that upon delivery of the ureteral stent of the properly selected length and then subsequent guidewire withdrawal, a proximal curled end of the ureteral stent will remain in the bladder.

In any embodiment, the visual indicia can include multiple bands of greater than 1 cm along the proximal end.

In any embodiment, the bands can be greater than 2 cm.

In any embodiment, the guidewire can be a metallic wire.

In any embodiment, the visual indicia can include multiple colors.

In any embodiment, a j-shaped curve can be at the distal end of the guidewire.

In any embodiment, the visual indicia can be along the proximal end of the guidewire.

In any embodiment, the visual indicia can be a color.

In another exemplary embodiment, the present invention is a system for guidewire placement of a ureteral stent comprising a guidewire configured for placement into a ureter of a patient via a scope, the guidewire having a length defined between a proximal end and a distal end, fluoroscopic indicia located between the ends in proximity to the distal end, and spaced a placement distance from the distal end, and visual indica located between the ends in proximity to the proximal end, and a ureteral stent configured to thread over the guidewire for delivery into a proper placement in the patient, and remain in the proper placement when the guidewire and scope are withdrawn, wherein the ureteral stent has a distal curled end configured to be located in the kidney upon the proper placement and a proximal curled end to be located in the bladder upon the proper placement, the fluoroscopic indicia aiding in the proper placement by providing a medical professional a fluoroscopic queue that the distal end of the guidewire is sufficiently located within the kidney, that upon delivery of the ureteral stent and then subsequent guidewire and scope withdrawal, the distal curled end of the ureteral stent will remain in the kidney, and the visual indica aiding in the proper placement by providing the medical professional a visual queue indicative of a properly selected length of the ureteral stent, that upon delivery of the ureteral stent of the properly selected length and then subsequent guidewire and scope withdrawal, the proximal curled end of the ureteral stent will remain in the bladder.

In any embodiment, the scope can be a cystoscope.

In any embodiment, the proper placement and properly selected length of the ureteral stent can be determined when the fluoroscopic indicia are located at the ureteropelvic junction and the visual indica is located at the ureterovesical junction.

In any embodiment, the visual indicia can be color.

In another exemplary embodiment, the present invention is a method comprising initiating insertion of a guidewire into a ureter of a patient, the guidewire having a length defined between a proximal end and a distal end, fluoroscopic indicia located between the ends in proximity to the distal end, and spaced a placement distance from the distal end, and visual indica located between the ends in proximity to the proximal end, receiving a fluoroscopic queue from the fluoroscopic indicia indicative that the distal end of the guidewire is sufficiently located within a kidney, ending insertion of the guidewire, receiving a visual queue from the visual indicia indicative of a properly selected length of a ureteral stent, threading a ureteral stent of the properly selected length over the guidewire, inserting, based upon the fluoroscopic indicia, a distal curled end of the ureteral stent into the kidney of the patient to provide a proper placement of the ureteral stent in the patient, and removing the guidewire from the ureter, wherein upon using the properly selected length of the ureteral stent, upon removal of the guidewire from the ureter, a proximal curled end of the ureteral stent is located in and remains in a bladder and the distal curled end of the ureteral stent remains in the kidney.

In another exemplary embodiment, the present invention is a guidewire for use with ureteral stents. The guidewire can have an elongated shape with a proximal end and a distal end. There can be a j-shaped curve at the distal end. The guidewire has indicia along the proximal end to indicate the length of the guidewire that was introduced into the patient. Based on the indicia or the visual inspection of the guidewire, the physician can select the appropriately sized stent to place in the patient.

In another exemplary embodiment, the present invention is a method for inserting a ureteral stent into the ureter in a patient comprising introducing into the ureter a guidewire having indicia at the proximal end of the guidewire into the patient, examining the indicia with respect to the patient, selecting a stent from a plurality of varying sized stents based on the amount of guidewire introduced into the patient as examined and as indicated by the indicia on the guidewire, and advancing the selected stent into the patient. The indicia are preferably defined color segments, which are easily visible to a person.

In another exemplary embodiment, the present invention is a guidewire and stent system having a plurality of stents of varying sizes and a guidewire. The stents are elongated, relatively flexible, and tubular members having at least one drainage opening extending through a wall thereof. The guidewire has a proximal end and distal end with a platinum marker 5 cm from the distal end and indicia on a proximal end to permit the proximal end to facilitate the placement of the stent member into the patient. The stent member receives the guidewire, and the stent member may be selected based on the indicia along the guidewire.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features can also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 illustrates a guidewire having indicia thereon.

FIG. 2 illustrates a stent insertable via the guidewire.

FIG. 3 illustrates an exemplary guidewire with indicia.

FIG. 4 illustrates the guidewire placed into a patient in which the indicia is visible to the physician.

FIG. 5 is a schematic isometric view illustrating the ureteral stent in position between the kidney and the bladder.

### DETAILED DESCRIPTION

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure,” and like characterizations, can include both being “at least substantially free” of something, or “at least substantially pure,” and being “completely free” of something, or “completely pure.”

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

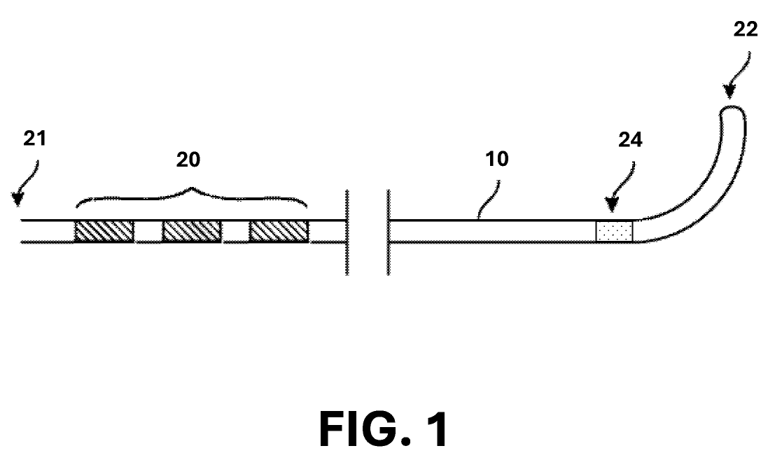

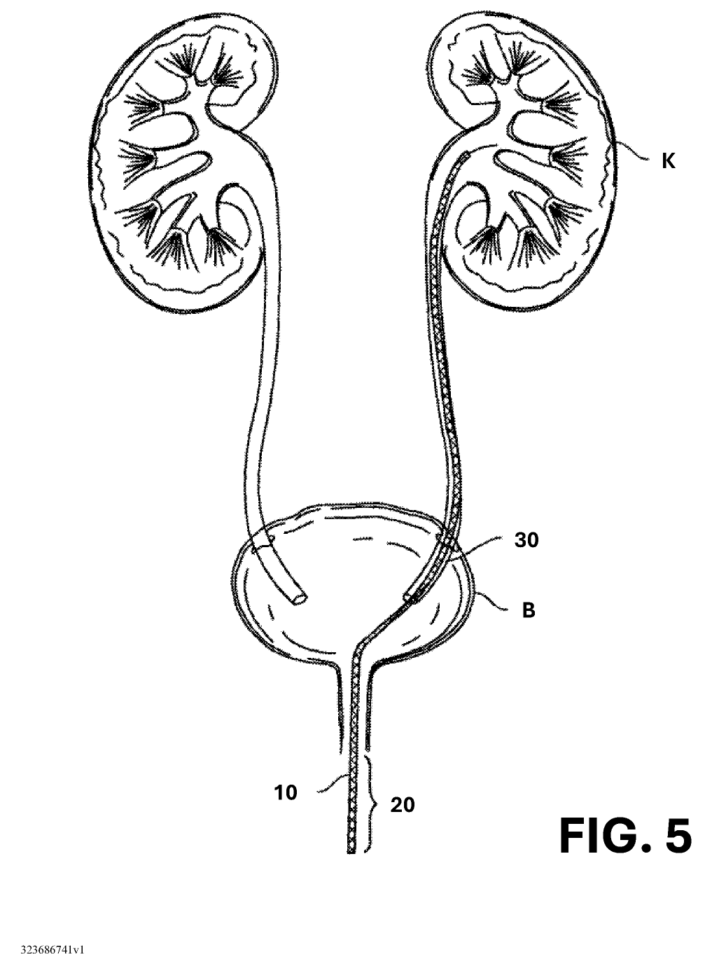

Ureteral stents 30 are small tubes inserted into the ureter to treat or prevent a blockage that prevents the flow of urine from the kidney K to the bladder B. A stent 30 is a small hollow tube that is placed into the ureter. During use, the ureteral stent 30 is installed by a physician using an elongated guidewire 10 that extends from the physician’s instrument known as a cystoscope, through the ureteral orifice, and then into the lumen of ureter until fluoroscopic indicia, preferably a platinum marker 24 on the guidewire 10 reaches the patient’s UPJ and kidney K, as shown in FIGS. 1 and 5. The physician inserts the uppermost end of the guidewire 10 and then selects the appropriately sized stent 30, based on the indicia 20, for use in the patient. When placed in the ureter, a top portion of the stent 30 has a small curl, usually about 5 cm, that sits in the kidney K and the opposite end curls in the bladder B.

FIG. 1 illustrates a guidewire 10 for placement of a ureteral stent 30. The guidewire 10 has a length defined between a proximal end 21 and a distal end 22. Visual indica 20 is located between the ends 21, 22 in proximity to the proximal end 21. Fluoroscopic indicia 24 is located between the ends 21, 22 in proximity to the distal end 22, and spaced a placement distance from the distal end 22.

The visual indica 20 aids in a proper placement of the stent 30 by providing a medical professional a visual queue indicative of a properly selected length for the ureteral stent 30, that upon delivery of the ureteral stent 30 of the properly selected length and then subsequent guidewire 10 withdrawal, a proximal curled end 31 of the ureteral stent 30 will remain in the bladder B.

The fluoroscopic indicia 24 aids in the proper placement of the ureteral stent 30 by providing the medical professional a fluoroscopic queue that the distal end 22 of the guidewire 10 is sufficiently located within the kidney K, that upon delivery of the ureteral stent 30 and then subsequent guidewire 10 withdrawal, a distal curled end 32 of the ureteral stent 30 will remain in the kidney K.

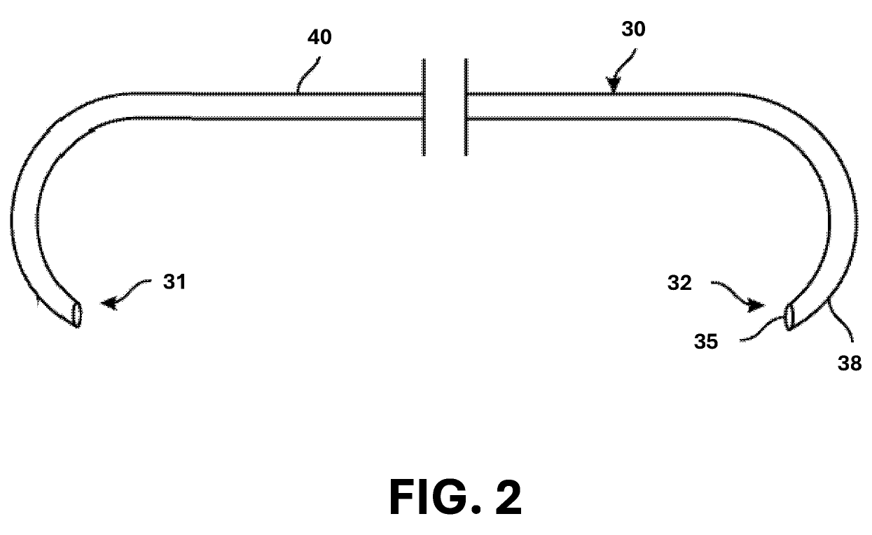

FIGS. 1-2 further illustrate an exemplary embodiment of the guidewire 10 having visible indicia 20 and the platinum marker 24 for determining the distance the guidewire 10 has been inserted into the ureter. The stent 30, currently available in an array of sizes (e.g., 22 cm, 24 cm, or 26 cm), comprises an elongated tubular member 35 having a relatively straight body portion 40 and opposed proximal end portion 31 and distal end portion 32. The distal end portion 32 is provided with a shape for retaining the distal end portion 38 in the kidney K. The proximal end portion 31 is provided with a shape for retaining the proximal end portion in the bladder B.

After or as the guidewire 10 is placed into the patient, the physician can select the appropriate sized stent 30 to place in the patient. The appropriate size will minimize patient discomfort and reduce the uncertainty for the physician.

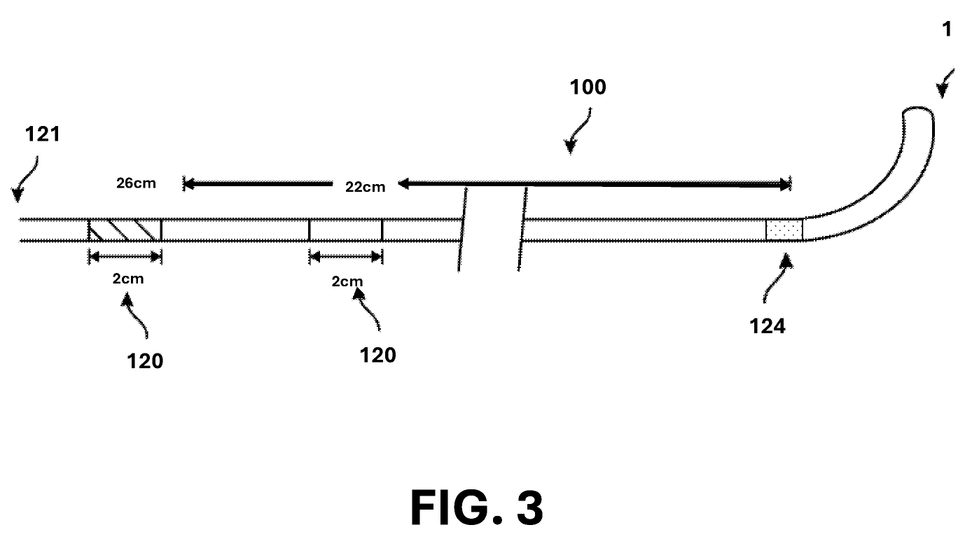

As can be seen from FIGS. 1 and 3, the guidewire 10, 100 has indicia 20, 120 at the proximal end 21, 121. The indicia 20, 120 can indicate the amount or the length of the guidewire 10, 100 that is introduced into the patient, and the appropriately sized stent 30 can be selected based on the length of the guidewire 10, 100 in the patient. The indicia 20, 120 preferably are colored bands, but can also be/include letters, numbers or any other indicator. The indicia 20 indicate the amount of the guidewire 10, 100 entering the patient. In an exemplary embodiment, the indicia can be a 2 cm band.

In an example shown in FIG. 3, the guidewire 100 has a radiopaque hydrophilic nitinol wire that is at the about 0.32 or 0.35 inches in diameter and 26 cm long. The total length of the guidewire 100 is about 240 cm, and the distal end 122 may be floppy for 5 cm. In this example, the visual indicia 120 includes a white 2 cm line/band in proximity of the proximal end 121 or tip of the floppy part of the guidewire 100. Other visual indicia 120 includes black and/or bright green, at 24 cm from proximal end and 26 cm from the proximal end. The fluoroscopic indicia 124 is located in proximity of the distal end 122 of the guidewire 100. A urologist or physician can then use the fluoroscopic indicia 124 as a fluoroscopic queue that the distal end 122 of the guidewire 100 is sufficiently located (in proper placement) within the kidney K, and the bands 120 used through visual inspection and can tell what length of stent 30 will best fit the length of ureter or patient. From the visual indicia 120, the physician can select an appropriately sized stent 30 for placement in the patient. Once the stent 30 is properly positioned, the guidewire 100 can be removed leaving the stent 30 in place.

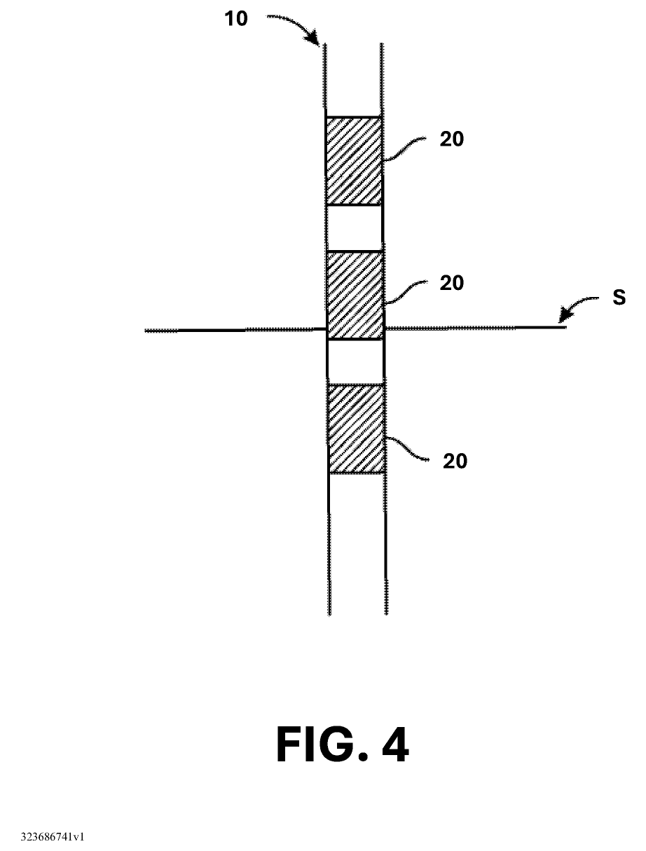

FIG. 4 illustrates an embodiment in which the guidewire 10 is introduced into a patient and in which a portion of the guidewire 10 is outside of the skin S of the patient for visual inspection. As can be seen, the number of indicia 20 visible to the physician is based on the size of the ureter of the patient. The physician then can select, based on professional judgment and the indicia, the appropriately sized stent 30 to place in the patient.

Another embodiment includes a simplified and more efficient catheterization/stent 30 placement procedure. As is further described below, certain embodiments disclosed herein may be capable of achieving various advantages, including by one or more of the following providing a physician with a plurality of multiple sized stents 30 or catheters for potentially placing into a patient, introducing a guidewire 10 with indicia 20 and a platinum marker 24 into the patient to a desired position, using the indicia to determine the length of stent 30 that optimally fit the patient or the ureter, and placing the selected sized stent 30 onto the guidewire 10 and ultimately in the patient. The physician selects the stent 30 or the size of the stent 30 based on the visual indicia on the guidewire 10. The stent 30 is normally inserted in the ureter by placing the distal end of the stent 30 onto the proximal end of the guidewire 10. Once the stent 30 is properly positioned, the guidewire 10 can be removed leaving the stent 30 in place.

In an exemplary embodiment, the physician has access to an array of varying sized stents 30. The ureteral stents 30 may have lengths varying from about 20 cm to about 30 cm and having outer diameters in the range of about 1 mm to about 4 mm. The outer diameters of the stents 30 may range from about 1.5 mm to about 3 mm and most preferably the stent 30 will have a wall thickness in the range of about 0.25 mm to about 1 mm. The stents 30 may be made of a suitable flexible material which is soft and stiff enough for the intended purpose and which may have a radiopaque material. The stent 30 may be supplied in various sizes and lengths. The physician selects the optimally sized stent 30 based upon his/her professional judgment and the indicia visible on the guidewire 10 after introduction into the patient. Ureteral stents 30 are known to those in the skill in the art and available for more than 40 years.

The ureteral stent 30 may optionally be radiopaque and/or as described above, have only a portion that is radiopaque (fluoroscopic indicia 24, 124). A radiopaque ureteral stent 30 is capable of having its placement confirmed by X-ray or fluoroscopy. The polymers that compose the ureteral stent 30 may be radiopaque, or the stent 30 may include radiopaque markers. For example, a ureteral stent 30 may include radiopaque markers at regular intervals along the length of the ureteral stent 30.

The ureteral stent 30 may optionally include a coating on the external portion of the ureteral stent 30. A hydrophilic coating may be used to improve patient comfort or to facilitate insertion. An antibacterial or antimicrobial coating, such as heparin, may be used to reduce infection or encrustation. The ureteral stent 30 may be coated with a single substance, or with a combination of multiple substances.

Specific embodiments generally relate to ureteral stents 30. By first proper placement of the guidewire 10 via indicia 24, and then visual review of indicia 20, the physician can select the appropriately sized stent 30 while reducing (compared with longer or shorter ureteral stents 30) patient discomfort when the stent 30 is placed within the patient’s body.

Methods for introducing a guidewire 10 or stent 30 into a patient are known to those of ordinary skill in the art. Various methods are used for inserting a ureteral stent 30 into a patient. In one example, the ureteral stent 30 is inserted though the urethral opening of a patient, advanced through the bladder and the ureter and subsequently placed in the kidney of the patient.

In certain embodiments of medical devices according to the invention, the stent 30 may have, for example, a circular cross-section. The stent 30 may have a substantially constant cross-sectional area along its length. Alternatively, the stent 30 may be tapered from about the distal end portion to about the proximal end portion with the size of the section decreasing from about 7 French to about 3 French. Other French sizes and tapering configurations are useful depending upon either the average size in a population or the actual size in an individual of the body structure or structures into which the device is placed.

The stent 30 may be constructed from any of a number of materials. Those materials that are useful include, for example, materials that are able to flex but also retain their shape, to a degree, when they are perturbed. Additionally, useful materials are, for example, materials that have a resilient quality, being able to regain at least some of their original shape when the stent 30 ceases to be perturbed and/or resist, for example, compression.

The ureteral stent 30 may be formed from a number of different biocompatible materials. The ureteral stent 30 may comprise one material or may be formed, for example by extrusion, of two or more materials along its length. For example, in an exemplary embodiment, the distal end portion of the ureteral stent 30 is formed from a first material having a first durometer and the proximal end portion is formed from a second material, which is softer and/or more flexible than the first material, having a second durometer different than the first durometer. Accordingly, the proximal end portion may be made of a softer or more flexible material than that of the distal end and/or the medial portion.

In an alternative embodiment, the indicia may be placed along the entire guidewire 10, so as to assist the physician in determining the amount of guidewire 10 being introduced into the patient.

Dimensions for the ureteral stent 30 of the present invention are not critical, however, they include internal diameters of 4.5 to 8.5 French, and lengths ranging from 20 cm to 32 cm.

Identical concerns that are mentioned above with respect to stents 30 are also applicable in the catheter arts, which include, without limitation intravenous catheters, guiding catheters, sheaths, umbilical catheters, trocar catheters, heart catheters (including, valvostomy catheters, angioplasty catheters, arthroscopy catheters, and the like), perfusion catheters, suction catheters, oxygen catheters, endoscopy catheters, endotracheal tubes, stomach tubes, feeding tubes, lavage tubes, rectal tubes, urological tubes, irrigation tubes, aneurysm shunts, stenosis dialators, trocars, and inserters, generally.

While the invention has been described in connection with specific embodiments thereof, it will be understood that it is capable of further modification, and this application is intended to cover any variations, uses, or adaptations of the invention following, in general, the principles of the invention and including such departures from the present disclosure as come within known or customary practice in the art to which the invention pertains and as may be applied to the essential features hereinbefore set forth, and as fall within the scope of the invention and the limits of the appended claims.

What is claimed is:

1. A guidewire for placement of a ureteral stent comprising:
a length defined between a proximal end and a distal end;
fluoroscopic indicia located between the ends in proximity to the distal end, and spaced a placement distance from the distal end; and
visual indica located between the ends in proximity to the proximal end;
wherein:
the fluoroscopic indicia aids in a proper placement of the ureteral stent by providing a medical professional a fluoroscopic queue that the distal end of the guidewire is sufficiently located within the kidney, that upon delivery of the ureteral stent and then subsequent guidewire withdrawal, a distal curled end of the ureteral stent will remain in the kidney; and
the visual indica aiding in the proper placement by providing the medical professional a visual queue indicative of a properly selected length for the ureteral stent, that upon delivery of the ureteral stent of the properly selected length and then subsequent guidewire withdrawal, a proximal curled end of the ureteral stent will remain in the bladder.

2. The guidewire of claim 1, wherein the fluoroscopic indicia comprises a platinum marker with a placement distance of 5 cm.

3. The guidewire of claim 1, wherein visual indicia comprises multiple bands of greater than 1 centimeter (cm) along a length of the guidewire in proximity to the proximal end.

4. The guidewire of claim 1, wherein the guidewire is a metallic wire.

5. The guidewire of claim 1, wherein the visual indicia comprises a color.

6. The guidewire of claim 1, wherein the visual indicia comprises multiple colors.

7. The guidewire of claim 1, the distal end of the guidewire comprises a 5 cm curve.

8. The guidewire of claim 3, wherein the bands are greater than 2 cm.

9. The guidewire of claim 1, wherein:
the fluoroscopic indicia comprises a platinum marker with a placement distance of 5 cm;
the visual indicia comprises multiple bands of greater than 1 cm along a length of the guidewire in proximity to the proximal end;
the guidewire is a metallic wire; and
the distal end of the guidewire comprises a 5 cm curve.

10. A system comprising:
the guidewire of claim 1 configured for placement into a ureter of a patient via a scope; and
a ureteral stent configured to:
thread over the guidewire for delivery into the proper placement in the patient; and
remain in the proper placement when the guidewire and scope are withdrawn;
wherein the ureteral stent has a distal curled end configured to be located in the kidney upon the proper placement and a proximal curled end to be located in the bladder upon the proper placement.

11. The system of claim 10, wherein the scope is a cystoscope.

12. The system of claim 10, wherein the proper placement and properly selected length of the ureteral stent is determined when:
the fluoroscopic indicia is located at the ureteropelvic junction; and
the visual indica is located at the ureterovesical junction.

13. The system of claim 10, wherein the visual indicia is color.

14. A method comprising:
initiating insertion of the guidewire of claim 1 into a ureter of a patient;
receiving the fluoroscopic queue from the fluoroscopic indicia indicative that the distal end of the guidewire is sufficiently located within the kidney;
ending insertion of the guidewire;
receiving the visual queue from the visual indicia indicative of the properly selected length of a ureteral stent;
threading the ureteral stent of the properly selected length over the guidewire;
inserting, based upon the fluoroscopic indicia, a distal curled end of the ureteral stent into the kidney of the patient to provide the proper placement of the ureteral stent in the patient; and
removing the guidewire from the ureter;
wherein upon using the properly selected length of the ureteral stent, upon removal of the guidewire from the ureter, a proximal curled end of the ureteral stent is located in and remains in the bladder and the distal curled end of the ureteral stent remains in the kidney.
